(19) United States
(12) Patent Application Publication
(10) Pub. No.: US 2009/0240440 A1
(43) Pub. Date: Sep. 24, 2009

(54) Non-Invasive Glucose Monitoring

(76) Inventors: Alex Shurabura, Jerusalem (IL); Tsvi Kan-Tor, Ramat-HaSharon (IL); Alexander Barkan, Beit-Shemesh (IL); Eitan Peled, Ramat-HaSharon (IL)

Correspondence Address: MARTIN D. MOYNIHAN d/b/a PRTSI, INC., P.O. BOX 16446, ARLINGTON, VA, 22215, US

(21) Appl. No.: 12/083,308

(22) PCT Filed: Oct. 18, 2006
(86) PCT No.: PCT/IL2006/001202

§ 371 Date: Apr. 9, 2008

(30) Foreign Application Priority Data
Oct. 20, 2005 (IL) 171491

(51) Int. Cl. G01N 33/48; G06F 19/00

(52) U.S. Cl. 702/19; 702/179

(57) ABSTRACT

A monitoring system for monitoring the glucose level of a subject having a glucose level history is disclosed. The system comprises (a) a non-invasive measuring device, operable to measure and record an electrical quantity from a section of the subject body, so as to provide a time-dependence of the electrical quantity over a predetermined time-period. The system further comprises (b) a processing unit, communicating with the non-invasive measuring device. The processing unit comprises: an extractor, for extracting a plurality of parameters characterizing the time-dependence, a correlation function calculator for calculate a subject-specific correlation function, and an output unit, communicating with the correlation function calculator and configured to output the glucose level of the subject. The subject-specific correlation function describes the glucose level history and is defined over a plurality of variables, each corresponding to a different parameter.

### FIELD AND BACKGROUND OF THE INVENTION

The present invention relates to glucose monitoring and, more particularly, to non-invasive glucose monitoring.

Diabetes mellitus is a widely distributed disease caused by either the failure of the pancreas to produce insulin or the body's inability to use insulin. Patients diagnosed with diabetes mellitus may suffer blindness, loss of extremities, heart failure and many other complications over time. In is recognized that there is no “cure” for the disease, but rather only treatment, most commonly with insulin injections in order to change the blood-glucose level.

To maintain a normal lifestyle, the diabetic patient must carefully and continuously monitor his or her blood glucose level on a daily, and oftentimes hourly basis. For example, blood glucose levels are critical in the maintenance and determination of cognitive functioning. With respect to the brain, blood glucose levels with respect to the brain influence and affect memory, awareness and attention. The consequences of reduced or elevated blood glucose levels on cognitive function are therefore more severe for subjects with poor glucose control such as individuals afflicted with diabetes. Hyperglycemia refers to a condition in which the blood glucose is too high, and the hyperglycemic subject is in danger of falling into coma. Hypoglycemia refers to a condition in which the blood glucose is too low, and the hypoglycemic subject is in danger of developing tissue damage in the blood vessels, eyes, kidneys, nerves, etc.

Foremost in the management of diabetes and the attainment of a successful insulin therapy is the need to continuously monitor the blood glucose level. Historically, this has been accomplished through painful, repetitive blood glucose tests requiring finger pricks three to four times daily. The primary reason for this regimen is that blood glucose levels fluctuate and stay out of balance until the next test or injection, and such fluctuations and imbalances greatly increase the risk of tissue and organ damage. The established method of glucose measurement expresses samples of blood onto a disposable test strip, and utilizes a meter device to read the test strip and report a quantitative blood glucose concentration. The appropriate dose of insulin is then calculated, measured and administered with a hypodermic needle.

Although highly accurate, this method requires drawing the patient's blood, which is less desirable than noninvasive techniques, especially for patients such as small children or anemic patients. The pain and inconvenience of the finger prick testing may be both physically and psychologically traumatic and oftentimes tend to discourage diabetics from adhering to the testing regimen as closely as they should. Thus, extensive research has been directed to develop techniques for monitoring blood glucose levels in a less invasive manner.

The difficulty in determining blood glucose concentration accurately may be attributed to several causes. First, blood glucose is typically found in very low concentrations within the bloodstream (e.g., on the order of 100 to 1,000 times lower than hemoglobin) so that such low concentrations are difficult to detect noninvasively, and require a very high signal-to-noise ratio. Second, there has been a lack of recognition of the kinds of noise and the proper method to use when removing this noise. Additionally, the optical characteristics of glucose are very similar to those of water which is found in a very high concentration within the blood. Thus, where optical monitoring systems are used, the optical characteristics of water tend to obscure the characteristics of optical signals due to low glucose concentration within the bloodstream.

In an attempt to accurately measure blood glucose levels within the bloodstream, several alternative methods have been used. One such method contemplates determining blood glucose concentration by means of urinalysis or some other method which involves pumping or diffusing blood fluid from the body through vessel walls. However, although less traumatic then blood drawing, acquiring urine samples is also inconvenient to the patient. Additionally, urinalysis is known to be less accurate than a direct measurement of glucose within the blood, since the urine, or other blood fluid, has passed through the kidneys.

Another proposed method of measuring blood glucose concentration is by means of optical spectroscopic measurement. In such devices, light of multiple wavelengths may be used to illuminate a relatively thin portion of tissue, such as a fingertip or an earlobe. A spectral analysis is then performed to determine the properties of the blood flowing within the illuminated tissue. Although such a method is highly desirable due to its noninvasive character and its convenience to the patient, problems are associated with such methods due to the difficulty in isolating each of the elements within the tissue by means of spectroscopic analysis. The difficulty in determining blood glucose concentration is further exacerbated due to the low concentration of glucose within blood, and the fact that glucose in blood has very similar optical characteristics to water. Thus, it is very difficult to distinguish the spectral characteristics of glucose where a high amount of water is also found, such as in human blood.

Following are several other techniques for non-invasive measurements of blood glucose.

U.S. Pat. No. 5,139,023 discloses a technique in which glucose diffuses across the buccal mucosal membrane into a glucose receiving medium, where the glucose is measured for correlation to determine the blood glucose level. The glucose receiving medium includes a permeation enhancer capable of increasing the glucose permeability across the mucosal membrane. U.S. Pat. No. 5,968,760 discloses a method for measuring blood glucose levels without separation of red blood cells from serum or plasma. U.S. Pat. No. 6,580,934 discloses a detection technique by inducing a time-varying temperature on a surface of the body, varying the temperature and then determining the glucose concentration based on the absorbance from radiation emitted from the surface of the body. U.S. Pat. No. 6,442,410 discloses a method for determining the blood glucose level based on an ocular refractive correction by measuring and then determining the ocular refractive correction to a database of known ocular refractive corrections and blood glucose concentrations. U.S. Pat. No. 6,477,393 discloses a technique that includes irradiating a surface of the subject by electromagnetic radiation and detecting the displaced radiation. The detection is then processed to provide blood glucose concentration. U.S. Pat. No. 6,565,509 discloses a transcutaneous electromechanical sensor which is responsive to an analyte enzyme and a sensor control unit for placement on skin that intermittently transmits data from analyte-dependent signals produced by the electromechanical sensor.

Attempts have also been made to correlate between electrical impedance parameters and the concentration of glucose in a blood of a patient. For example, Russian Patent No. 2,073,242 discloses a method of indicating the sugar concentration in the blood based on the change of the dielectric permittivity of a finger placed in an electric field. Russian Patent No. 2,088,927 teaches that glucose concentration definition is obtained according to the reactive impedance variation. U.S. Pat. No. 5,792,668 presents glucose measurement using radio frequency electromagnetic components at frequencies in the 2 GHz to 3 GHz range and provides a measure of combined concentration of glucose and NaCl. The examination includes analysis of the effective complex impedance presented by the specimen and effective phase shift between the transmitted and reflected signal at the specimen. U.S. Pat. No. 6,841,389 discloses glucose measurement using measurements of the total impedance of the skin of a patient and linear model of a first order correlation between the glucose concentration and the total impedance.

The major problem with presently known non-invasive glucose monitoring techniques is that these techniques are inferior to the invasive methods from the standpoint of measurement accuracy. Specifically, a considerable percentage (more than 20%) of glucose predictions obtained by presently known non-invasive glucose monitoring techniques do not fall within the so called “A zone” of a standard Clarke Error Grid, which is typically defined as a zone in which the predicted glucose levels are close to actual blood glucose levels. In several non-invasive techniques, glucose predictions also fall within the “C”, “D” or “E” zones of the Clarke Error Grid, which are typically defined as the zones in which the predictions significantly deviate from the reference values and treatment decisions based on such predictions may well be harmful to a patient.

Additionally, currently available glucose monitors suffer from the limitations of high operating cost and difficulty in use. Conventional hand-held instruments for home use fail in that the instruments do not consistently provide the correct assessment of blood glucose concentration over the entire length of time the instruments are used. These hand-held devices are calibrated with a one-time global modeling equation hard-wired into the instrument, to be used by all patients from time of purchase onward. The model does not provide for variations in the unique patient profile which includes such factors as gender, age or other existing disease states.

There is thus a widely recognized need for, and it would be highly advantageous to have a method and system for non-invasive glucose monitoring, devoid of the above limitations.

### SUMMARY OF THE INVENTION

According to one aspect of the present invention there is provided a method of determining a subject-specific correlation function correlating an electrical quantity characterizing a section of a subject body to a glucose level of the subject. The method comprises: non-invasively measuring the electrical quantity, so as to provide a time-dependence of the electrical quantity over a predetermined time-period; measuring the glucose level of the subject a plurality of times, thereby providing a series of glucose levels; using the time-dependence for extracting a plurality of parameters characterizing the time-dependence; and performing a statistical analysis so as to correlate the series of glucose levels to at least one of the plurality of parameters; thereby determining the subject-specific correlation function.

According to another aspect of the present invention there is provided a method of estimating the glucose level of a subject having a glucose level history. The method comprises calculating a subject-specific correlation function describing the glucose level history, and using the subject-specific correlation function for estimating the glucose level of the subject.

According to yet another aspect of the present invention there is provided a method of monitoring the glucose level of a subject having a glucose level history. The method comprises: non-invasively measuring an electrical quantity from a section of the subject body so as to provide a time-dependence of the electrical quantity over a predetermined time-period; using the time-dependence for extracting a plurality of parameters characterizing the time-dependence; calculating a subject-specific correlation function describing the glucose level history; and using the subject-specific correlation function for estimating the glucose level of the subject; thereby monitoring the glucose level of the subject.

According to further features in preferred embodiments of the invention described below, the subject-specific correlation function is defined over a plurality of variables, each variable of the plurality of variables corresponding to a different parameter of the plurality of parameters.

According to still further features in the described preferred embodiments the variables are respectively weighted by a plurality of subject-specific coefficients.

According to still further features in the described preferred embodiments at least one variable of the plurality of variables is powered by a subject-specific power.

According to still further features in the described preferred embodiments the method further comprises testing the accuracy of the subject-specific correlation function according to a predetermined accuracy criterion, and, if the predetermined accuracy criterion is not satisfied then updating the subject-specific correlation function.

According to still further features in the described preferred embodiments the method further comprises updating the subject-specific correlation function at least once.

According to still further features in the described preferred embodiments the updating is of at least one of the variables, subject-specific coefficients and subject-specific powers.

According to still further features in the described preferred embodiments the updating comprises: measuring the glucose level of the subject a plurality of times, thereby providing a series of glucose levels; and performing a statistical analysis so as to correlate the series of glucose levels to at least one of the parameters and to provide an updated plurality of variables and an updated plurality of subject-specific coefficients.

According to still another aspect of the present invention there is provided a system for determining a subject-specific correlation function. The system comprises: (a) a glucose level input unit configured for receiving a series of glucose levels; (b) a non-invasive measuring device operable to measure and record the electrical quantity, so as to provide a time-dependence of the electrical quantity over a predetermined time-period; and (c) a processing unit communicating with the non-invasive measuring device, and comprising: (i) an extractor, communicating with the non-invasive measuring device and being operable to extract a plurality of parameters characterizing the time-dependence; and (ii) a correlating unit, communicating with the extractor and being supplemented with statistical analysis software configured to correlate the series of glucose levels to at least one of the plurality of parameters, thereby to determine the subject-specific correlation function.

According to an additional aspect of the present invention there is provided apparatus for estimating the glucose level of a subject having a glucose level history. The apparatus comprises: a correlation function calculator, operable to calculate a subject-specific correlation function describing the glucose level history, and to estimate the glucose level of the subject based on the subject-specific correlation function; and an output unit, communicating with the correlation function calculator and configured to output the glucose level of the subject.

According to yet an additional aspect of the present invention there is provided a monitoring system for monitoring the glucose level of a subject having a glucose level history. The system comprises a non-invasive measuring device and a processing unit, communicating with the non-invasive measuring device. The processing unit comprises: an extractor, a correlation function calculator, and an output unit. The output unit communicates with the correlation function calculator and configured to output the glucose level of the subject.

According to further features in preferred embodiments of the invention described below, the system further comprises a display for displaying glucose level of the subject.

According to still further features in the described preferred embodiments the system further comprises an updating unit designed and configured for updating the subject-specific correlation function at least once.

According to still further features in the described preferred embodiments the updating unit comprises: a glucose level input unit; and a correlating unit being supplemented with statistical analysis software configured to correlate the series of glucose levels to at least one of the plurality of parameters and to provide an updated plurality of variables and an updated plurality of subject-specific coefficients.

According to still further features in the described preferred embodiments the updating unit is a component in the processing unit.

According to still further features in the described preferred embodiments the display is attached to the processing unit.

According to still further features in the described preferred embodiments the display is attached to the non-invasive measuring device.

According to still further features in the described preferred embodiments the non-invasive measuring device and the processing unit are encapsulated by or integrated in a first housing.

According to still further features in the described preferred embodiments the non-invasive measuring device is encapsulated by or integrated in a first housing and the processing unit is encapsulated by or integrated in a second housing.

According to still further features in the described preferred embodiments the first housing is sized and configured to be worn by the subject on the body section.

According to still further features in the described preferred embodiments the apparatus or system comprises an alert unit configured to generate a sensible signal when the glucose level is below a predetermined threshold.

According to still further features in the described preferred embodiments the alert unit is configured to generate a sensible signal when the glucose level is above a predetermined threshold.

According to still further features in the described preferred embodiments the alert unit is configured to generate a sensible signal when a rate of change of the glucose level is above a predetermined threshold.

According to still further features in the described preferred embodiments the alert unit is configured to generate a sensible signal when the glucose level increases.

According to still further features in the described preferred embodiments the alert unit is configured to generate a sensible signal when the glucose level decreases.

According to still further features in the described preferred embodiments the system further comprises at least one communication unit, wherein the non-invasive measuring device is configured to transmit data through the at least one communication unit.

According to still further features in the described preferred embodiments the predetermined time-period is correlated to a heart rate of the subject.

According to still further features in the described preferred embodiments the predetermined time-period equals at least a heart beat cycle of the subject.

According to still further features in the described preferred embodiments the predetermined time-period equals an integer number of heart beat cycles of the subject.

According to still further features in the described preferred embodiments the predetermined time-period is continuous.

According to still further features in the described preferred embodiments the predetermined time-period is discontinuous.

According to still further features in the described preferred embodiments the electrical quantity comprises electrical impedance characterizing the body section.

According to still further features in the described preferred embodiments the non-invasive measuring device comprises: a plurality of surface contact electrodes; a generator configured for generating signals and transmitting the signals to at least two of the plurality of surface contact electrodes; and an impedance detector configured for detecting the electrical impedance.

According to still further features in the described preferred embodiments at least one of the parameters comprises a value of the electrical quantity at a transition point on the time-dependence.

According to still further features in the described preferred embodiments at least one of the parameters comprises a ratio between two values of the electrical quantity, the two values corresponding to different transition points on the time-dependence.

According to still further features in the described preferred embodiments at least one of the parameters comprises a difference between two values of the electrical quantity, the two values corresponding to different transition points on the time-dependence. According to still further features in the described preferred embodiments the value is normalized by a time-constant, the time-constant being extracted from the time-dependence.

According to still further features in the described preferred embodiments at least one of the parameters comprises a time-interval corresponding to a transition point on the time-dependence.

According to still further features in the described preferred embodiments at least one of the parameters comprises a time-derivative of the time-dependence.

According to still further features in the described preferred embodiments at least one of the parameters comprises an average time-derivative of at least a segment of the time-dependence.

According to still further features in the described preferred embodiments at least one of the parameters comprises a slope along a segment of the time-dependence.

According to still further features in the described preferred embodiments wherein the transition point is selected from the group consisting of a maximal systolic point, a minimal systolic point, a maximal diastolic point, a minimal diastolic point, a minimal incisures point, myocardial tension start point and myocardial tension end point.

The present embodiments successfully address the shortcomings of the presently known configurations by providing a method, apparatus and system which can provide accurate and reliable non-invasive glucose level monitoring.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flowchart diagram of a method for determining a subject-specific correlation function, according to various exemplary embodiments of the present invention;

FIG. 2 illustrates a representative example of a time-dependence of an electrical impedance, according to various exemplary embodiments of the present invention;

FIG. 3 is a schematic illustration of a system for determining a subject-specific correlation function, according to various exemplary embodiments of the present invention;

FIG. 4 is a flowchart diagram of a method for monitoring the glucose level of a subject, according to various exemplary embodiments of the present invention;

FIG. 5 is a schematic illustration of a monitoring system for monitoring the glucose level of the subject, according to various exemplary embodiments of the present invention;

FIGS. 6a-b are schematic illustrations of two alternative embodiments for the system, where in FIG. 6a the system is manufactured as a single unit and in FIG. 6b system is manufactured as two or more separate units;

FIG. 7 is a schematic electronic diagram for the monitoring system, according to various exemplary embodiments of the present invention;

FIGS. 8-10 show comparisons between glucose levels estimated according to the teachings of the present embodiments, and glucose levels measured invasively, for three different subjects; and

FIG. 11 is a scatter plot superimposed on a Clarke Error Grid, showing reference glucose levels versus glucose level as estimated according to various exemplary embodiments of the present invention.

### DESCRIPTION OF THE PREFERRED EMBODIMENTS

The present embodiments comprise a method and system which can be used for monitoring the glucose level of a subject. Specifically, the embodiments can be used for non-invasive glucose monitoring using a subject-specific correlation function.

The principles and operation of a method and system according to the present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present embodiments exploit changes of electrical properties of biological material over time for the purpose of estimating the glucose level of a subject. Without being bound to any theory it is assumed that the electrical properties of a section of the human body may depend, inter alia, on the concentration of glucose in the blood present in the body section. At the same time, it is recognized that the electrical properties are also affected by other factors, including, for example, the viscosity of the blood, drugs that may be present in the blood or other tissue components, blood flow, blood volume, presence of plaque and others. Yet, the characteristic time scale for a change in the electrical properties differs from one factor to the other. In particular, since fluctuations in glucose concentration occur over a relatively short time scale, the characteristic time scale for a change in the electrical properties when the change is due to such fluctuation is also short. Conversely, fluctuations in the other factors affecting the electrical characteristics occur on a much larger time scales (from days to months).

Hence, while conceiving the present invention it has been hypothesized and while reducing the present invention to practice it has been realized that a correlation can be established between the electrical characteristics of a body section and the glucose concentration, provided the correlation is established based on measurements performed over a sufficiently short time period.

The present inventor has thus discovered a method and system for determining a subject-specific correlation function, which correlates between an electrical quantity characterizing a section of a subject body and the glucose level of the subject. The subject-specific correlation function can then be used for estimating the glucose level of the subject at a later time. Specifically, once determined, the subject-specific correlation function can be used for non-invasive monitoring of the glucose level of the subject. Preferably, the subject-specific correlation function is updated from time to time so as to account for factors affecting the electrical properties over larger time scales.

As demonstrated in the Examples section that follows, the technique discovered by the present Inventor allow accurate and reliable non-invasive glucose level monitoring.

The term “accurate and reliable monitoring” as used herein, refers to monitoring procedure in which at least 90%, more preferably at least 95%, most preferably essentially all (say above 99.5%) the estimated glucose levels are within the so called “A zone” and “B zone” of a standard Clarke Error Grid. Of the points falling in the “A zone” and “B zone” of a standard Clarke Error Grid, at least 85%, more preferably at least 88%, more preferably at least 90%, even more preferably at least 92%, say about 95% or more of the estimated glucose levels fall within the “A zone” of a standard Clarke Error Grid. It is understood that like any analytical technique, calibration validation and recalibration are required for the most accurate operation.

The term “Clarke Error Grid”, as used herein, is a broad term and is used in its ordinary sense, including, without limitation, an error grid analysis, which evaluates the clinical significance of the difference between a reference glucose level and an estimated glucose level, taking into account the relative difference between the estimated and reference levels, and the clinical significance of this difference. See W. Clarke, D. Cox, L. Gonder-Fredrick, W. Carter and S. Pohl, “Evaluating clinical accuracy of systems for self-monitoring of blood glucose”, Diabetes Care 1987; 10:622-628, which is incorporated by reference herein in its entirety.

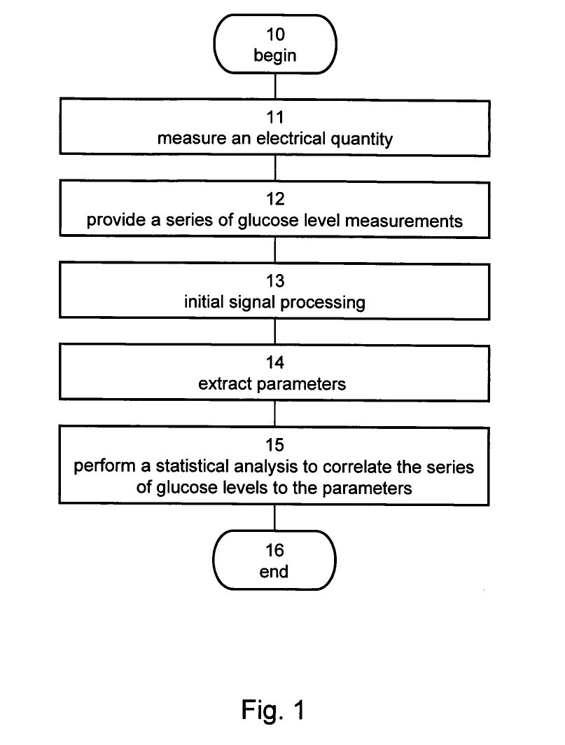

Referring now to the drawings, FIG. 1 is a flowchart diagram of a method for determining a subject-specific correlation function, according to various exemplary embodiments of the present invention.

It is to be understood that, unless otherwise defined, the method steps described hereinbelow can be executed either contemporaneously or sequentially in many combinations or orders of execution. Specifically, the ordering of the flowchart diagrams is not to be considered as limiting. For example, two or more method steps, appearing in the following description or in the flowchart diagrams in a particular order, can be executed in a different order (e.g., a reverse order) or substantially contemporaneously. Additionally, several method steps described below are optional and may not be executed.

The method begins at step 10 and continues to step 11 in which an electrical quantity is non-invasively measured. The electrical quantity is preferably measured on the surface of the body section, such as, but not limited to, arm, leg, chest, waist, ear and any portion thereof. Any electrical quantity which is indicative of at least a few electrical properties of the selected section of the body, and which therefore characterizes the section can be measured. Representative examples include, without limitation, impedance, reactance, resistance, voltage, current and any combination thereof.

Measurements of such and other electrical quantities are known in the art and typically involve application of output electrical signals to the surface of the body section and detection of input electrical signals from the surface. Thus, two or more surface contact electrodes are preferably connected to the exterior surface of the body section, and the output electrical signals are transmitted via the electrodes to the surface. Typically, the output electrical signals comprise alternating voltage at a frequency of several tens of KHz. A preferred frequency range is, without limitation, from about 20 KHz to about 50 KHz, more preferably from about 30 KHz to about 35 KHz.

As used herein the term “about” refers to ±10%.

In various exemplary embodiments of the invention the parameters of the output electrical signal (frequency, voltage) are constant over the period of measurement, but varying parameters (e.g., a first frequency over a first time-interval, a second frequency over a second time-interval, etc.), are also contemplated.

When more than two surface electrodes are employed in the measurement, they are preferably paired either statically or dynamically. In the embodiment in which dynamic paring is employed, each electrode is dynamically assigned to another electrode, according to all possible pairing combinations or according to any subset thereof. Thus, when there are N electrodes (N>2), there are N/(N−1) possible pairs, and the paring includes at least a few of these pairs. Thus, in a preferred embodiment in which there are four electrodes, there are 12 possible electrode pairs. Use of dynamic pairing is preferred when the placement of the electrodes is not done by a trained technician. In the embodiment in which static pairing is employed, the pairs are selected in advance. For example, in a preferred embodiment in which there are four electrodes, the first electrode can be paired to the second electrode and the third electrode can be paired to the fourth electrode.

The measurement of the electrical quantity is performed to obtain a time-dependence of the electrical quantity over a predetermined time period. Ideally, the measurement of the electrical quantity is continuous resulting in a continuous set of values of the electrical quantity over a continuous time interval. However, such continuous set of values is rarely attainable, and in practice, although the measurement can be continuous, a plurality of values of the electrical quantity is recorded at a plurality of discrete time instances. The number of recorded samples is nevertheless sufficient for obtaining (e.g., by interpolation) the time-dependence of the electrical quantity over a predetermined time period. Thus, a sequence of samples of the electrical quantity is generated at various time-instances separated from each other by sufficiently short time-intervals. The obtained time-dependence is a mathematical function Z(t) which expresses the value of the electrical quantity as a function of time t, for at least a few instances within the predetermined time period [t₁, t₂]. More preferably, the mathematical function is a continuous function expressing the value of the electrical quantity as a function of time, for any time t∈[t₁, t₂].

The predetermined time-period is, as stated, sufficiently short so as to allow correlating the electrical quantity to the glucose level, substantially without “contaminating” the correlation with contributions of factors other than glucose level. Typically, but not obligatorily, the predetermined time-period is correlated with the heart rate of the subject. In various exemplary embodiments of the invention the time-period equals at least a heart beat cycle of the subject. For example, the time period can equal one a heart beat cycle or an integer number of heart beat cycles.

The time period can be either continuous or discontinuous. For example, the electrical quantity can be measured over several consecutive heart beat cycle or the measurement can be stopped for a certain time-interval and continued thereafter. The measurement can also be performed without stopping, but several measurements can be discarded during their analysis for improving the quality of the results. In this case, the time period can effectively be discontinuous. According to a preferred embodiment of the present invention at least a few cycles of measurements are taken over several heart beat cycles and are then averaged, by any averaging procedure, to provide a time-dependence of the electrical quantity over a single heart beat cycle.

According to a preferred embodiment of the present invention two or more cycles of measurements are performed. Thus, measurement cycles can be performed at different hours of the day, over a period of several hours, a day or more. Thus, several time-dependences of the electrical quantity are obtained, one time-dependence for each measurement cycle. Preferably, the measurement cycles are performed at parts of the day in which glucose level fluctuations are expected. For example, measurement cycles can be performed before and after each meal during the day. One or more measurement cycle can also be performed during long intervals between meals.

The method continues to step 12 in which the glucose level of the subject is measured a plurality of times to provide a series of glucose levels. This step can be executed by any glucose measuring technique, device or system. Preferably, the glucose level measurement provides real (non-estimated) blood glucose levels. Thus, a blood sample of the subject is placed in a suitable device, such as a blood analyzer, which measures and displays the glucose concentration in the blood sample. A representative example of a glucose measuring system is the FreeStyle™ blood glucose monitoring system which is commercially available from Abbott Laboratories, Illinois, U.S.A. Also contemplated is the Accu-Check® glucose meter, any of the HemoCue® Glucose Systems, Roche Cobas Mira® analyzer and Kodak Ektachem® Analyzer.

It is expected that during the life of this patent many relevant glucose measuring systems will be developed and the scope of the term glucose measuring device is intended to include all such new technologies a priori.

The measurement of glucose level of the subject is preferably synchronized with the measurement of the electrical quantity, so as to allow correlating the electrical quantity with the glucose level, as further detailed hereinbelow. Preferably, at least one time-dependence of the electrical quantity is obtained for each measurement of glucose level. Thus, each measurement of glucose level preferably corresponds to a sequence of electrical quantity measurements.

In various exemplary embodiments of the invention the method proceeds to step 13 in which the obtained sequence of electrical quantity measurements is subjected to an initial signal processing, such as, but not limited to, Fourier transform, fast Fourier transform, autocorrelation processing, wavelet transform and the like. The purpose of the initial processing is to delineate the components of the mathematical function at a particular domain and to allow removing the undesired components from further processing. For example, a Fourier, fast Fourier or wavelet transform can be used to delineate the various frequency components of the time-dependence, and to remove those frequency components identified as noise. Subsequently, an inverse transform can be applied so as to present the electrical quantity in the time domain.

The method continues to step 14 in which a plurality of parameters are extracted from the time-dependence of the electrical quantity. According to a preferred embodiment of the present invention many parameters are extracted so as to optimize the construction of the correlation function, as further detailed hereinafter. A preferred number of parameters is, without limitation, at least 4, more preferably at least 6, more preferably at least 8, more preferably at least 10, more preferably at least 12, more preferably at least 14, more preferably at least 16 parameters characterizing the time-dependence.

When the several cycles of electrical measurements are taken and several time-dependences are obtained, each parameter is a vector quantity having a sequence of entries, one entry for each time-dependence. For example, measurement cycles can be taken over several (not necessarily consecutive) heart-beat cycles, such that a time-dependence is obtained for each heart-beat cycle. In this embodiment, each parameter is a vector having one entry for each heart-beat cycle.

The parameters may comprise, for example, the heart rate, the total value of the electrical quantity (e.g., maximal value relative to zero), values of the electrical quantity at transition points on the time-dependence (one value per transition point) and the like. Generally, a transition point is identified on the time-dependence of the electrical quantity as points in which a functional transition occurs.

As used herein “functional transition” refers to any detectable mathematical transition of a function, including without limitation, a transition of a given function (e.g., a change of a slope, a transition from increment to decrement or vice versa) and a transition from one characteristic functional behavior to another (e.g., a transition from a linear to a nonlinear behavior or a transition from a first nonlinear behavior to a second, different, nonlinear behavior).

The functional transitions can be identified, for example, by calculating a derivative of the time-dependence and finding zeros thereof. As will be appreciated by one of ordinary skill in the art, a transition of a function can be characterized by a zero of one of its derivatives. For example, a transition from increment to decrement or vice versa is characterized by a zero of a first derivative, a transition from a concave region to a convex region or vice versa (points of inflection) is characterized by a zero of a second derivative, etc. According to a preferred embodiment of the present invention any derivative of the time-dependence can be used. Generally, the functional transitions are preferably characterized by a sign inversion of an nth derivative of the time-dependence, where n is a positive integer.

Additionally or alternatively, the functional transitions can be identified by observing deviations of the time-dependence from smoothness. In this embodiment, the functional transitions can be identified either with or without calculating the derivatives of the time-dependence. For example, deviations from smoothness can be identified by comparing the time-dependence to a known smooth function.

In various exemplary embodiments of the invention at least a few of the transition points are associated with different stages of the cardiac cycle. Representative examples for transition points suitable for the present embodiments, include, without limitation, points associated with systole (maximal and/or minimal amplitude of the systolic wave), points associated with diastole (maximal and/or minimal amplitude of the diastolic wave), points associated with incisures (local minimum), points associated with myocardial tension (myocardial tension start point and myocardial tension end point), and the like.

The parameters can also comprise one or more ratios between two values of the electrical quantity. For example, a parameter can be extracted by dividing the value of the electrical quantity at one transition point by the value of the electrical quantity at another transition point. Additionally or alternatively, the parameters can also comprise one or more differences between two values of the electrical quantity. In this embodiment, a parameter can be extracted by subtracting the value of the electrical quantity at one transition point from the value of the electrical quantity at another transition point. Thus, according to the presently preferred embodiment of the invention the parameters comprise at least one interval along the ordinate of the time-dependence.

Any extracted parameter can be normalized to provide another parameter. Preferably, the parameter is normalized by a time-constant which is also extracted from time-dependence. For example, in various embodiments of the invention the parameters are normalized to the duration of a heart beat. As will be appreciated by one of ordinary skill in the art, such normalization procedure can double the number of parameters, whereby each parameter can have a normalized and non-normalized value.

Another type of parameters which is contemplated relates to the calculations of time-intervals. For example, a parameter can be a time-interval which corresponds to a transition point. Such time-interval can be calculated by subtracting a predetermined time-reference from the time corresponding to the particular transition point. The predetermined time-reference can be, for example, the beginning of the heart beat cycle. Also contemplated are parameters which represent time-interval between two transition points. Thus, according to the presently preferred embodiment of the invention the parameters comprise at least one interval along the abscissa.

An additional type of parameters which is contemplated is time-derivative of the time-dependence. Thus, the derivative of the time-dependence can be used both indirectly and directly for extracting parameters. Indirectly, the derivative is used for identifying transition points at which various parameters can be obtained or calculated. Directly, the derivative itself is used as a parameter. In various exemplary embodiments of the invention the derivative is used in both ways. Firstly, the transition point is identified and secondly the value of the derivative at the identified transition point is stored as one of the parameters.

Alternatively or additionally, an average time-derivative of one or more segment of the time-dependence can be calculated and stored as a parameter. For example, one parameter can be the average derivative of the time-dependence at a segment associated with the systolic wave. When an average first-derivative is calculated, it can be conveniently expressed as a slope along the respective segment, which slope can be expressed in terms of an angle.

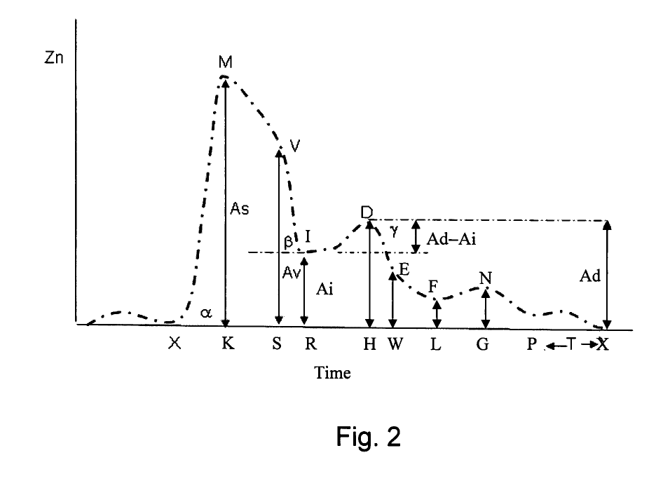

FIG. 2 illustrates a representative example of a time-dependence Zₙ(t) of the electrical quantity in the preferred embodiment in which the electrical quantity is the electrical impedance, Zₙ. Shown in FIG. 2 are various transition points and parameters. The transition points on Zₙ(t) include, point of maximum of the systolic wave (M), point of minimum of the systolic wave (V), point of minimum level of the incisures (I), point of maximum amplitude of the diastolic and top of the dicrotic wave (D), point of inflection (E), point of local minimum (F), and point of local maximum (N). Also shown in FIG. 2 are representative points along the abscissa, including the beginning point of the fast blood supply in the wrist (X), the time of maximum of the systolic wave (K), the time of minimum of the systolic wave (S), the time of minimum level of the incisures (R), the time of maximum amplitude of the diastolic (H), the time of inflection point E(W) the time of local minimum point F(L), the time of local maximum point N(G), and the beginning point of the tension myocardium period (P).

Several representative parameters are marked on FIG. 2. These include, maximal amplitude of the systolic wave (As), minimal amplitude of the systolic wave (Av), amplitude of the incisures (Ai), amplitude of the diastolic wave (Ad), the period of the tension myocardium (T), the difference between the amplitude of the diastolic wave and the amplitude of the incisures (Ad−Ai), the angle of slope of the ascending segment of the systolic wave (α), the angle of slope of the descending segment of the systolic wave (β), and the angle of slope of the descending segment of the diastolic wave (γ). As stated, many other parameters can be extracted. Thus, for example, Thus, for example, parameters by calculating the following intervals along the ordinate: EW, FL, NG, EW−FL, NG−FL, ±(NG−EW), Av−Ai, Ad−EW, etc. Parameters can also be extracted by calculating the following time-interval along the abscissa: XX, XK, XS, XH, HX, XV, XR, HP and the like. Additional parameters can be extracted by calculating various ratios (e.g., As/Ad, As/Av, As/Ai), differences (e.g., As−Ad, As−Av, As−Ai) and various normalized quantities (e.g., As/XX, Ad/XX, Ai/XX).

When the measurements of the electrical quantity are taken over several heart-beat cycles, one or more parameters, as extracted from one heart-beat cycle, can be compared to the respective parameters as extracted from other heart-beat cycles. This comparison can serve as a “quality” control, whereby heart-beat cycles from which one or more of the extracted parameters do not satisfy a predetermined goodness criterion are discarded from the following analysis.

Once the parameters are extracted, the method continues to step 15 in which a statistical analysis is performed so as to correlate the series of glucose levels to at least one of the extracted parameters. Any statistical analysis procedure can be employed for the correlation, include, without limitation, linear regression, polynomial regression, non-linear regression, exponential fit and the like. The statistical analysis is preferably implemented using a data processor, such as an electronic device having digital computer capabilities (e.g., an Advanced RISC Machine), supplemented with a suitable algorithm. The correlation between the series of glucose levels and the extracted parameters is expressed as a correlation function which is preferably defined over a plurality of variables weighted by a plurality of coefficients. Mathematically, the correlation function can be expressed as the following function

F(X₁, X₂, . . . )=a₀+a₁X₁^{y1}+a₂X₂^{y2}+ . . . ,

where, X₁, X₂, . . . are the variables of F, a₀, a₁, a₂, . . . are constant coefficients, and y₁, y₂, . . . are constant powers. When y₁=y₂= . . . =1, F is a linear function, but this need not necessarily be the case because for some subjects a non-linear function, in which at least one of the powers differs from 0 or 1, may be more suitable than a linear function.

In any event, each variable X of the correlation function corresponds to one of the parameters which are extracted from the time-dependence of the electrical quantity. Since the measurements of the electrical quantity and the glucose level measurements are performed for the same subject, the obtained correlation function F, and in particular its coefficients, a₀, a₁, a₂, etc. and optionally also the powers y₁, y₂, etc., is subject-specific. Optionally and preferably, the combination of variables X₁, X₂, . . . are also subject-specific. In other words, for different subjects the combination of variables may correspond to different extracted parameters.

Since, as stated, each parameter is preferably a vector with one entry for each time-dependence, the statistical analysis can be performed separately for each vector. Thus, in one substep, a statistical analysis is performed to correlate the first parameter to the series of glucose levels; in another substep, a statistical analysis is performed to correlate the second parameter to the series of glucose levels, and so on. In various exemplary embodiments of the invention a correlation test is applied for each statistical analysis and parameters for which a predetermined correlation criterion is not met are preferably discarded from the correlation function, or, equivalently, are weighted by a zero coefficient. The degree of correlation of each parameter can be quantified, for example, by calculating one or more statistical moments (e.g., Pearson product-moment correlation, also known as “R²-value”) or goodness-of-fit (e.g., χ²-test, Kolmogorov test, etc.) which characterizes the correlation. Based on the statistical moment, goodness-of-fit or the like, a correlation score is preferably assigned for each parameter, where high correlation score corresponds to strong (positive or negative) correlation and low correlation score corresponds to weak or no correlation. The correlation criterion can be that the parameter is discarded if the correlation score is below a predetermined threshold. The correlation criterion can be global or it can also be specific to the subject.

Once statistical analyses are performed to all the extracted parameters, an additional statistical analysis is preferably performed to the parameters for which the correlation criterion is met, so as to provide a multi-variable subject-specific correlation function. The purpose of the additional analysis is to determine the value of the coefficient of each parameter to a better accuracy. Any type of analysis can be employed, e.g., using matrix manipulation and the like. The additional analysis can also comprise a regression procedure as known in the art.

The additional analysis can be performed simultaneously or, more preferably, iteratively, e.g., according to the correlation score of the parameters in descending order. A global correlation score is preferably calculated so as to quantify the correlation between the subject-specific correlation function and the series of glucose levels. When the additional analysis is performed iteratively, the correlation score is preferably calculated during the iterative process. Such procedure allows monitoring the convergence rate of the process. The global correlation score can also serve for defining a stopping criterion for the iteration. For example, the iterative process can be continued until the global correlation score is above a predetermined threshold. Alternatively, the iterative process can continue for all the parameters.

The method ends at step 16.

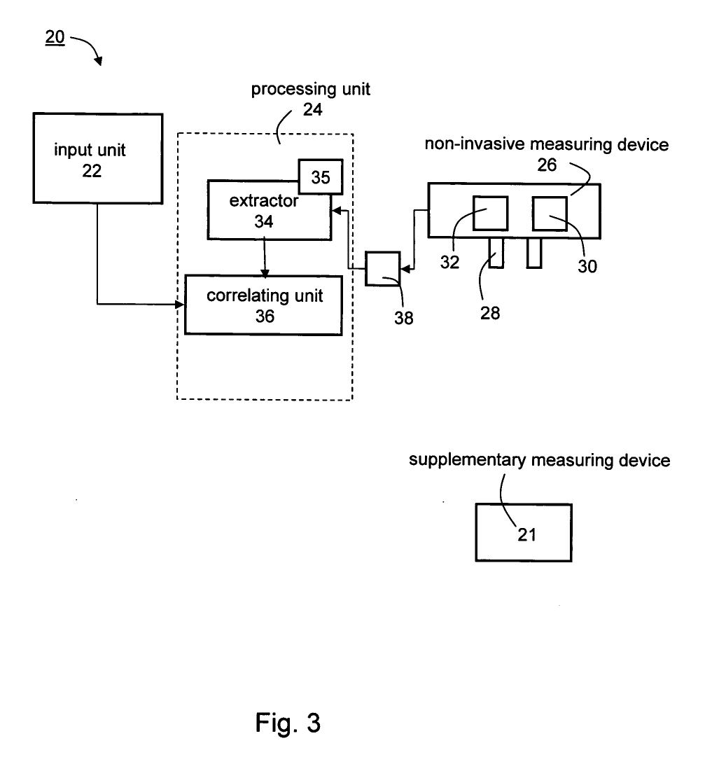

Reference is now made to FIG. 3 which is a schematic illustration of a system 20 for determining a subject-specific correlation function, according to various exemplary embodiments of the present invention.

System 20 comprises a glucose level input unit 22, configured for receiving a series of glucose levels. The glucose levels can be measured using a supplementary measuring device, such as a blood analyzer and the like as described above. The supplementary measuring device is generally shown at 21. The glucose levels can be inputted to unit 22 either manually or automatically by establishing direct or indirect communication between the glucose measuring device and unit 22. System 20 further comprises a non-invasive measuring device 26 which measures and records the electrical quantity, to provide the time-dependence of electrical quantity. In various exemplary embodiments of the invention device 26 comprises a plurality of surface contact electrodes 28, a generator 30 for generating the output signals and transmitting them to electrodes 28, and a detector 32 for detecting input signals from electrodes 28.

According to the preferred embodiment of the present invention, electrodes 28 are porous (e.g., of a partially sintered metallic aggregate, or the like). This provides greater skin contact and also results a better signal to noise ratio for the measurement of the electrical quantity. Alternatively, electrodes 28 can comprise a graphite surface portion which serves as a porous active-electrical contact-member of the electrode. In the preferred embodiment in which the electrical quantity is electrical impedance, generator 30 can generates alternating voltage and detector 32 can be configured to detect impedance, is commonly known in the art.

System 20 further comprises a processing unit 24, communicating with device 26. Unit 24 serves for processing the electrical quantity values measured by device 26 and for correlating the electrical quantity to the series of glucose levels. Thus, unit 24 is preferably designed and configured to execute at least a few of method steps 13-15 described above. Calculations performed by unit 24 can be executed by a set of computer instructions for performing the calculations. Such set of computer instructions can be embodied in on a tangible medium such as a computer. The set of computer instructions can also be embodied on a computer readable medium, comprising computer readable instructions for carrying out the calculations. In can also be embodied in electronic device having digital computer capabilities (e.g., an Advanced RISC Machine) arranged to run the computer instructions on the tangible medium or execute the instructions on a computer readable medium.

The communication between device 26 and system 20 can be directly, in which case device 26 and unit 24 are preferably encapsulated by or integrated in the same housing, or via a communication unit 38, in which case device 26 and unit 24 can be encapsulated by separate housings.

In various exemplary embodiments of the invention processing unit 24 comprises an extractor 34, which communicates with device 26 and is programmed to extract the parameters from the time-dependence as described above. Extractor 34 can also be programmed to perform the initial processing step described above.

Extractor 34 preferably receives from device 26 the time-dependence Z(t) as a plurality of values of the electrical quantity respectively associated with a plurality of discrete time instances. Such input to extractor 34 is sufficient for calculating any of the aforementioned parameters. Extractor 34 preferably comprises a locator 35 for locating transition points of Z(t) as further detailed hereinabove (see, e.g., points M, V, I, D, E, F, N in FIG. 2). Thus, in various exemplary embodiments of the invention locator 35 calculates one or more mathematical derivatives of Z(t) with respect to the time and finds zeroes of the mathematical derivatives, to thereby locate the transition points. Locator 35 can also locate other points on the curve of Z(t), such as end points, points of deviation from smoothness and the like.

Unit 24 further comprises a correlating unit 36, which is in communication with extractor 34 and which is supplemented with statistical analysis software configured to correlate the glucose levels to one or more of the parameters, as further detailed hereinabove.

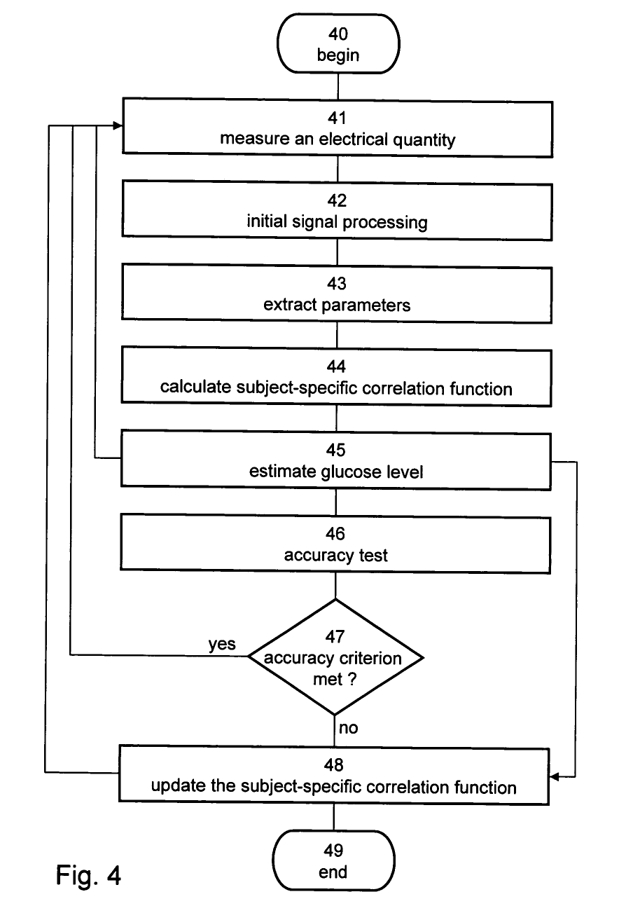

Reference is now made to FIG. 4 which is a flowchart diagram of a method for monitoring the glucose level of a subject, according to various exemplary embodiments of the present invention. Broadly speaking, the method measures electrical quantity on the surface of the subject's body and estimate the glucose level of the subject based on a subject-specific correlation function, which describes the glucose history of the subject, and which can be determined, e.g., using then flowchart diagram of FIG. 1 and/or system 20.

Thus, the method begins at step 40 and continues to step 41 in which the electrical quantity (e.g., impedance, reactance, resistance, voltage, current, etc.) is non-invasively measured, to provide the time-dependence of the electrical quantity, as further detailed hereinabove. Optionally and preferably, the method continues to step 42 in which initial processing is performed, as further detailed hereinabove. The method continues to step 43 in which a plurality of parameters are extracted from the time-dependence of the electrical quantity. The number of parameters which are extracted depends on the number of variables of the subject-specific correlation function. This number can be significantly smaller than the number of parameter which are needed to be extracted for the purpose of determining the correlation function, because, as stated, one or more coefficients of the correlation function can be zero.

The method continues to step 44 in which the subject-specific correlation function F(X₁, X₂, . . . ) is calculated. The calculation of F is performed by respectively substituting the values of the extracted parameters as the variables of the function, and utilizing the values of the coefficients and powers for obtaining the value of F. Once the value of F is known the level of glucose in the blood of the subject can be estimated. Typically, the value of F equals the value of glucose level. Alternatively, a normalization step is employed for translating the value of F to glucose level.

The method can then loop back to step 41 to continue the monitoring. The monitoring loop can be repeated one or more times, as desired. In various exemplary embodiments of the invention after a few such monitoring loops and/or after a certain time period (not to be confused with the period associated with the time-dependence of the electrical quantity), the method continues to step 46 in which the accuracy of the subject-specific correlation function is tested.

The accuracy test is preferably performed by comparing the estimated glucose level to the actual blood glucose level. Thus, in various exemplary embodiments of the invention a blood sample of the subject is preferably placed in a suitable blood analyzer which measures and displays the glucose level in the blood sample. The estimated glucose level at the time the blood sample was taken is then compared to the reading of the analyzer.

Such accuracy testing can be performed every 10-20 monitoring loops, once a day, every other day, once a week, etc. For different subjects a different accuracy testing regimen can be set. Preferably, the accuracy testing regimen is determined based on the accumulated experience regarding the glucose estimates for the specific subject. For example, accuracy testing can be performed for a particular subject every, say, 10 monitoring loops, for a period of one week, and, depending on the outcome of these tests, the physician or the subject can determine whether or not such accuracy testing regimen is sufficient. Thus, if the accuracy of the estimated glucose level is sufficient, e.g., during the entire week, the accuracy testing rate can be set to once a week; if the accuracy of the estimated glucose level is sufficient, during a part of the week, the accuracy testing rate can be set to once every such part of the week; if, on the other hand the accuracy of the estimated glucose level is insufficient, after each such accuracy test, the accuracy testing rate is preferably increased.

The method continues to decision step 47 in which the method decides whether or not an accuracy criterion is met. The accuracy criterion can be a sufficiently small deviation of the estimated from the non-estimated glucose level. Thus, the method calculates the deviation of the estimated from the non-estimated glucose level and compares the deviation to a predetermined threshold. The threshold can be set according to the Food and Drug Administration (FDA) criterion. For example, the threshold can be set to about 20% deviation or less.

In the accuracy criterion is satisfied (for example, if the deviation is below the threshold), the method can loop back to step 41. If the accuracy criterion is not satisfied, the method proceeds to process step 48 in which the subject-specific correlation function is updated. Yet, the method can also proceed to step 48 even without executing the accuracy test (step 46).

The update of the subject-specific correlation function is preferably in accordance with the principles described above, and is preferably performed using elements of system 20 and/or by executing one or more of method steps 10-16. Any part of the subject-specific correlation function can be updated. Specifically, any variable (i.e., the number and/or type of parameters which are utilized for constructing the multi-variable function), coefficient and/or power can be updated.

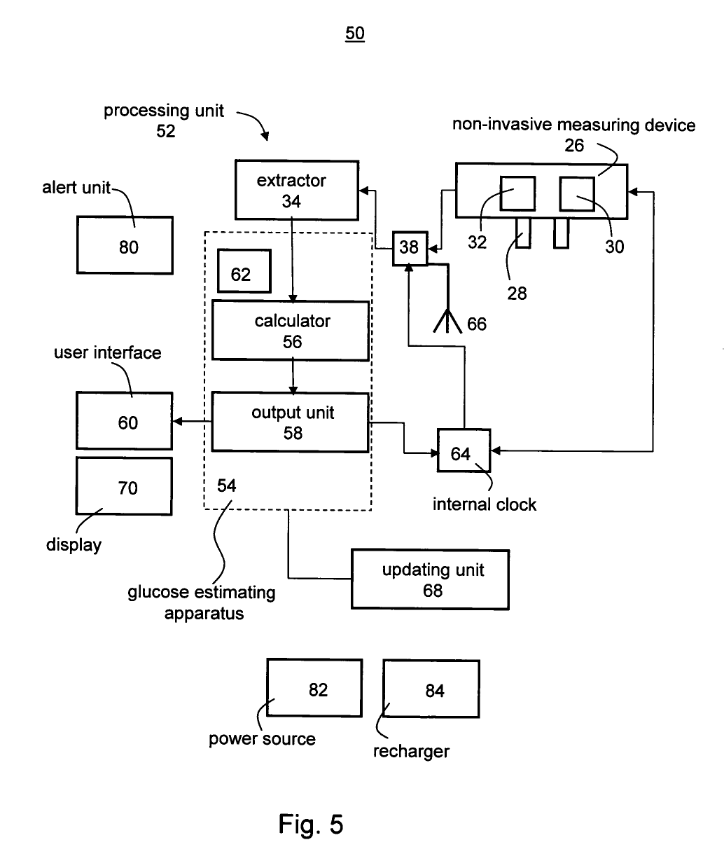

Reference is now made to FIG. 5 which is a schematic illustration of a monitoring system 50 for monitoring the glucose level of the subject, according to various exemplary embodiments of the present invention. System 50 comprises non-invasive measuring device 26, and a processing unit 52 which preferably communicates with device 26, e.g., via communication unit 38, as described above. Unit 52 serves for processing the electrical quantity values measured by device 26 and for calculating the subject-specific correlation function F(X₁, X₂, . . . ), which describes the glucose history of the subject, and which can be determined, e.g., using then flowchart diagram of FIG. 1 and/or system 20.

Thus, unit 52 is preferably designed and configured to execute at least a few of method steps 42-44 described above. Calculations performed by unit 52 can be executed by a set of computer instructions for performing the calculations as described above.

Unit 52 comprises extractor 34 which extracts the parameters from the time dependence as further detailed in connection with system 20 hereinabove. Unit 52 further comprises a glucose estimating apparatus 54 which estimates the glucose level of the subject. In various exemplary embodiments of the invention apparatus 54 comprises a correlation function calculator 56 which calculates the subject-specific correlation function F(X₁, X₂, . . . ) and estimates the glucose level of the subject based on the value of F(X₁, X₂, . . . ). Thus, apparatus 54 preferably comprises memory media 62 which store in a readable format the coefficients and powers characterizing the subject-specific correlation function. Memory media 62 can store a zero coefficients for variables corresponding to parameters which do not contribute to the value of F. Alternatively, memory media 62 can store the list of parameters which contribute to the value of F.

Apparatus 54 preferably comprises an output unit 58, which communicates with calculator 56 and configured to output the glucose level of the subject. In various exemplary embodiments of the invention system 50 comprises a user interface 60 for displaying the estimated glucose level and optionally additional information such as, but not limited to, temporal data (time and date) associated with the estimates to the user of system 50. The information is preferably in a format which is readable, or otherwise detectable and decipherable, by the user. Device 60 can be configured to present a message in any of a number of modes, include, without limitation, visual (such as a text message or a flashing light), audible (such as a series of beeps or audible speech) and mechanical (such as vibrations). One or more of these modes can allow device 60 to provide a physically impaired user with the estimated glucose level. Preferably, device 60 comprises a display 70, such as, but not limited to, a liquid crystal display. Display 70 can be attached to processing unit 52, non-invasive measuring device 26, or it can be provided as a separate unit.

The estimates of glucose level can additionally or alternatively be transmitted by communication unit 38 over a wireless or wired communication network 66. The estimates of glucose levels, as well as temporal data (time and date) associated with the estimates, can be stored in memory media 62 or they can be transmitted communication network 66 to a remote location.

According to a preferred embodiment of the present invention system 50 comprises an updating unit 68 designed and configured for updating the subject-specific correlation function as described above. Thus, unit 68 can comprise, or can be operatively associated with system 20 or selected elements thereof. Optimally and preferably, unit 68 comprises supplementary measuring device 21 for measuring the glucose concentration as further detailed hereinabove. According to a preferred embodiment of the present invention at least one part of unit 68 is a component in processing unit 52. For example, since extractor 34 of system 20 function essentially as the extractor of system 50, extractor 34 can also be used by unit 68. Additionally, input unit 22 and/or correlating unit 36 can be installed as components in unit 68.

According to a preferred embodiment of the present invention system 50 comprises an internal clock 64. This is particularly useful for obtaining the temporal data. Clock 64 can also be used for timing the measurements performed by device 26, according to a regimen set, e.g., by the physician. As an accessory, clock 64 can communicate with display 70 to allow the temporal data to be displayed.

According to a preferred embodiment of the present invention system 50 further comprises an alert unit 80 which generates a sensible (visual, audible or mechanical) signal to the user. Unit 80 is preferably configured to alert in at least one of the following events: glucose level which is above a predetermined threshold, glucose level which is below a predetermined threshold, rate of change of the glucose level which is above a predetermined threshold, increasing glucose level, and decreasing glucose level.

System 50 can further comprise at least one power source 82 for supplying energy to its components, e.g., unit 52 and device 26 and other components which may be employed. Power source 82 is preferably portable, and can be replaceable or rechargeable, integrated with, or being an accessory to system 50. Power source preferably provides a voltage of less than 15 volts, e.g., from about 1.5 volts to about 9 volts, and a current of the order of a micro-Ampere, e.g., from about 0.1 μA to about 2 μA. Representative examples include, without limitation a solar power source, a mobile a voltage generator, an electrochemical cell, a traditional secondary (rechargeable) battery, a double layer capacitor, an electrostatic capacitor, an electrochemical capacitor, a thin-film battery (e.g., a lithium cell), a microscopic battery and the like. In embodiments in which power source 82 is rechargeable, system 50 preferably comprises a recharger 84, which can be integrated with or supplied separately to system 50 as desired.

The various components of system 50 can be assembled into one compact housing or, alternatively, system 50 can be manufactured as separate units.

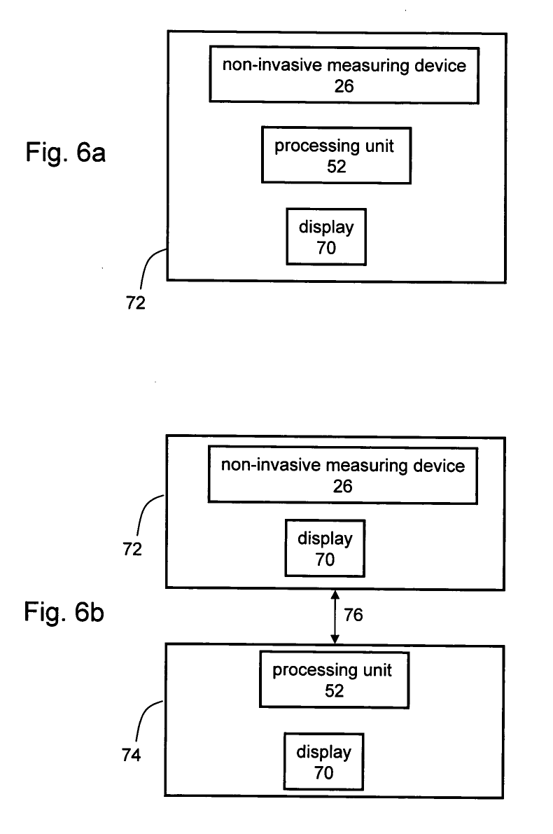

Reference is now made to FIGS. 6a-b which are schematic illustrations of two alternative embodiments for system 50. In the embodiment illustrated in FIG. 6a, non-invasive measuring device 26, processing unit 52 and optionally display device 70 are encapsulated by or integrated in a housing 72. In this embodiment all the communication between the various elements of system 50 is internal and preferably via wired communication channels. In the embodiment illustrated in FIG. 6b, non-invasive measuring device 26 is encapsulated by or integrated in a housing 72 and processing unit 52 is encapsulated by or integrated in a separate housing 74. In this embodiment any one of housing 72 and housing 74 can include display 70. The communication between the components in housing 72 and the components in housing 74 can be via communication channel 76, which can be wireless (e.g., Wi-Fi®, Bluetooth®) or wired as desired. When a wired communication channel is used, the communication wires are preferably detachable.

Housing 72 is preferably sized and configured to be worn by the subject on the body section. For example, housing 72 can be in the form of a watch device or the like which is configured to be worn about the wrist of the user. The term “watch device” as used herein refers to any type of device which is configured to be worn about the wrist of the user, and which does not necessarily include, but does not specifically exclude, a time-keeping function.

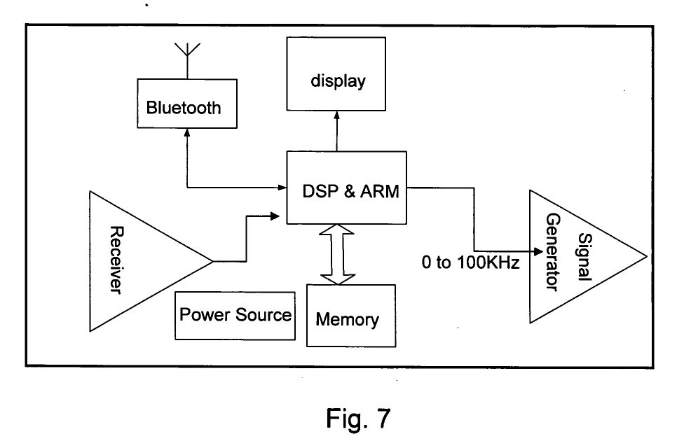

A schematic electronic diagram for monitoring system according to various exemplary embodiments of the present invention is illustrated in FIG. 7. The diagram shows a central control unit having a digital signal processing unit (DSP) and an Advanced RISC Machine (ARM), a signal generator and a receiver. The signal generator is fed by the central control unit and transmits output signals at the desired frequency via the contact electrodes (not shown, see FIGS. 3 and 5). Receiver feeds the central control unit by input signals received from the electrodes. Also shown is a memory media which communicates with the central control unit. The central unit can read from the memory media the coefficients and powers of the subject-specific function, and it can also write to the memory media information such as the estimated glucose level and temporal data associated therewith. The central control unit can also provide the information to a display which in turn displays the information in a readable, or otherwise detectable and decipherable format. Additionally or alternatively the central control unit can transmit the information, e.g., over a Bluetooth® network or the like.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

### Example 1

### Determination of Subject-Specific Correlation Function

The teachings of the present embodiments have been used for determining subject-specific correlation functions in three different subjects.

### Methods

The following protocol was used for each subject:

(i) 10 measurements of glucose levels were taken invasively using FreeStyle™ blood glucose monitoring system. The measurements were taken before and after meals, at intervals of 10-20 minutes between consecutive measurements. The obtained glucose levels were recorded as the reference glucose history of the subject.

(ii) Electrical impedance was measured on the wrist of the subject. 10 cycles of measurements were performed synchronously with the invasive glucose level measurements. For each cycle of electrical impedance measurements, the time-dependence of the electrical impedance was obtained over a heart-beat cycle. Thus, a 10 time-dependence of the electrical impedance were obtained.

(iii) For each time-dependence, the following parameters were extracted (see FIG. 2 and accompanying description hereinabove): Base (total impedance (relative to zero), As, heart rate (Pulse per Minute), T, β, XS, α, HP, NG, γ, Ad, EW, Ad−Ai, As/Ad, As/XX, As/Av, As/Ai, XH and HX. Since there were 10 time-dependences, each extracted parameter was a vector quantity with 10 entries, one for each time-dependence.

(iv) A statistical analysis was performed to correlate each parameter to the glucose levels measured at step (i) above, and a correlation score was assigned for each parameter. The parameters with highest scores were identified and other parameters were marked as not correlative.

(v) Additional statistical analysis was performed to construct a subject-specific correlation function F in which the variables correspond to the parameters with highest correlation scores. In the present example, linear algebra technique was employed, and F was a linear function of its variables (all powers were set to 1). The linear algebra technique assigned a coefficient for each variable, while each of the other parameters was assigned with a zero coefficient. The linear algebra technique also resulted in a free constant which was added to the function F.

(vi) The deviating of F from the to the glucose levels measured at step (i) above as well as the standard deviation and the correlation score associated with F were calculated.

(v) 10 additional cycles of measurements of the electrical impedance were taken, similarly to step (ii). For each such additional measurement, the glucose level was estimated using the now-known subject-specific correlation function.

(vi) 10 measurements of reference glucose levels were taken invasively using FreeStyle™ blood glucose monitoring system. The measurements were taken at the times of the additional cycles of step (v) and were compared to the estimated values.

### Results

### Subject No. 1

Table 1 below summarize the glucose history, the entries of each (vector) parameter and the calculated correlation score of each parameter.

TABLE 1

| | Time | | | | | | | | | | |
|---|---|---|---|---|---|---|---|---|---|---|---|
| Parameter | 0 | 20 | 01:00 | 01:20 | 01:40 | 02:00 | 02:20 | 02:40 | 03:00 | 03:20 | score |
| Base | 205 | 199 | 169 | 169 | 169 | 170 | 171 | 172 | 174 | 172 | −0.65 |
| As | 29.5 | 35 | 32.5 | 38 | 44.5 | 38 | 36 | 36 | 33 | 36.5 | 0.45 |
| heart rate | 61 | 62 | 60 | 59 | 60 | 60 | 59 | 58 | 57 | 58 | −0.60 |
| T | 16 | 10 | 28 | 14.5 | 6 | 20 | 31 | 2.5 | 6 | 7 | 0.03 |
| β | 9.5 | 11.8 | 12.7 | 16.7 | 12.2 | 10.9 | 10.4 | 19 | 10.1 | 9 | 0.24 |
| XV | 26.6 | 25.5 | 28 | 22.6 | 31.2 | 36.2 | 32 | 18.2 | 30.2 | 34 | 0.15 |
| α | 5.7 | 4.2 | 5.85 | 5.65 | 8.95 | 7.7 | 8.6 | 4.85 | 8.3 | 7.15 | 0.59 |
| HP | 25.5 | 38.3 | 27.1 | 30.85 | 30.9 | 30.1 | 25.2 | 28.1 | 24.5 | 30.75 | −0.43 |
| NG | 62.5 | 43.5 | 66 | 69 | 62 | 56 | 66 | 71 | 70 | 50 | 0.51 |
| γ | 9.65 | 7.9 | 10.9 | 12.8 | 13.65 | 10.2 | 10.2 | 14.2 | 9.9 | 9.3 | 0.51 |
| Ad | 18.75 | 21.95 | 22 | 26.65 | 23.75 | 20.3 | 20.15 | 26.45 | 19.6 | 19.4 | 0.19 |
| EW | 61.4 | 59.4 | 58.35 | 40.95 | 29.8 | 53 | 67.8 | 26.6 | 77 | 62.7 | −0.24 |
| Ad-Ai | 2.1 | 0.7 | 3 | 1.1 | 1.1 | 1.4 | 1.2 | 2.2 | 1.7 | 1.5 | −0.13 |
| As/Ad | 162.7 | 159.7 | 150 | 142.6 | 187.3 | 187.2 | 182.55 | 137.7 | 166.7 | 190.7 | 0.24 |
| As/XX | 30.8 | 50.6 | 31.7 | 35.8 | 43.9 | 41.3 | 34.95 | 32.1 | 30.8 | 45.7 | −0.25 |
| As/Av | 111.1 | 104.45 | 111.5 | 106.1 | 111.7 | 121.65 | 114.65 | 105.6 | 114.6 | 121.7 | 0.28 |
| As/Ai | 185 | 182.95 | 196.4 | 153.8 | 201.8 | 273.45 | 221.5 | 145.7 | 229.45 | 274.1 | 0.16 |
| XH | 24 | 18.55 | 25.6 | 26.95 | 25.6 | 23 | 23.9 | 31.45 | 24 | 19.7 | 0.49 |
| HX | 73.5 | 50.05 | 75.2 | 76.8 | 73.6 | 67.5 | 78.1 | 78.1 | 82.3 | 60.3 | 0.56 |
| reference glucose | 115 | 102 | 118 | 147 | 163 | 173 | 195 | 184 | 161 | 139 | |
| history | | | | | | | | | | | |

The criterion for the calculation of F was that no more than two values of F will deviate from the reference glucose history by more than 20%. For this subject, two parameters with highest scores were identified: Base with a correlation score of −0.65 and α with a correlation score of 0.57. The following correlation function was obtained for subject No. 1:

F(Base,α)=178.579−0.61953Base+10.851α

Table 2 below displays the deviating of F from the reference glucose history.

TABLE 2

| | reference | | | estimated | | |
|---|---|---|---|---|---|---|
| Time | glucose history | Base | α | glucose | Δ | Δ [%] |
| 0 | 115 | 205 | 5.7 | 113 | −2 | −1.7% |
| 20 | 102 | 199 | 4.2 | 101 | −1 | −1.0% |
| 01:00 | 118 | 169 | 4.2 | 119 | 1 | 0.8% |
| 01:20 | 147 | 169 | 5.65 | 135 | −12 | −8.2% |
| 01:40 | 163 | 169 | 8.95 | 171 | 8 | 4.9% |
| 02:00 | 173 | 170 | 7.7 | 157 | −16 | −9.2% |
| 02:20 | 195 | 171 | 8.9 | 169 | −26 | −13.3% |
| 02:40 | 184 | 171 | 10.4 | 185 | 1 | 0.5% |
| 03:00 | 161 | 174 | 8.3 | 161 | 0 | 0.0% |
| 03:20 | 139 | 172 | 7.15 | 150 | 11 | 7.9% |

The corresponding standard deviation and correlation factor are 15.8 and 0.753, respectively. As shown no estimate exceeded the predetermined limit of 20%.

Table 3 below presents the values of the parameters Base and α as extracted from the time-dependences obtained from 10 additional cycles of measurements performed for subject No. 1. The right column of Table 3 presents the glucose level as estimated according to the teachings of the present embodiments based on the reference glucose history of subject No. 1 (see Table 1) using the correlation function which is specific to subject No. 1.

TABLE 3

| Time | Base | α | estimated glucose |
|---|---|---|---|
| 0 | 203 | 5.5 | 112 |
| 20 | 169 | 3.15 | 108 |
| 01:00 | 169 | 6.2 | 141 |
| 01:20 | 170 | 6.3 | 142 |
| 01:40 | 170 | 8.75 | 168 |
| 02:00 | 172 | 11.4 | 196 |
| 02:20 | 171 | 7.9 | 158 |
| 02:40 | 172 | 9 | 170 |
| 03:00 | 171 | 7.6 | 155 |
| 03:20 | 171 | 6.35 | 142 |

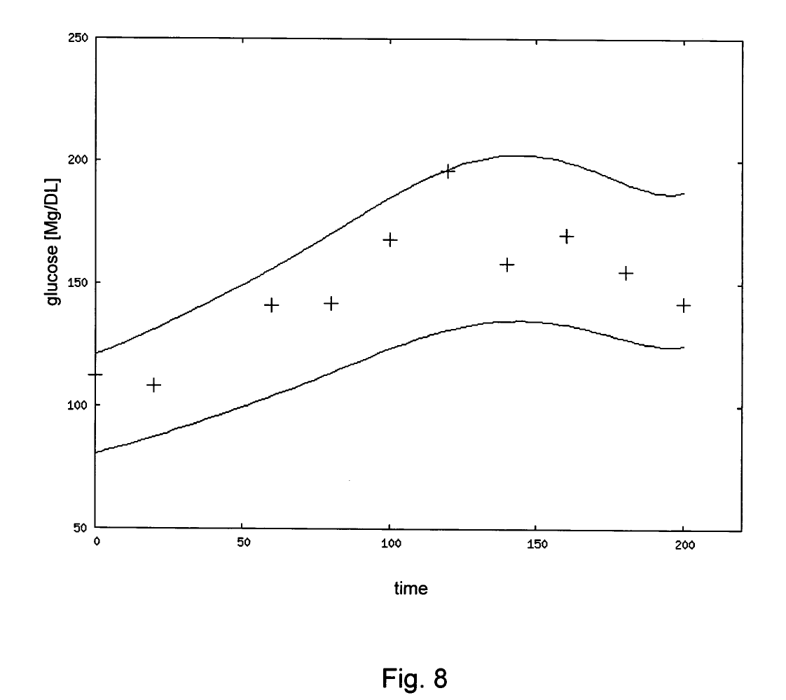

Table 4 below and FIG. 8 compare between the glucose levels of Table 3 as estimated according to the teachings of the present embodiments, and glucose levels measured invasively. The reference glucose levels in Table 4 were not used in the determination of the correlation function.

TABLE 4

| | reference | estimated | | |
|---|---|---|---|---|
| Time | glucose | glucose | Δ | Δ % |
| 0 | 101 | 112 | −11 | −11% |
| 20 | 106 | 108 | −2 | −2% |
| 01:00 | 134 | 141 | −7 | −5% |
| 01:20 | 128 | 142 | −14 | −11% |
| 01:40 | 166 | 168 | −2 | −1% |
| 02:00 | 167 | 196 | −29 | −17% |
| 02:20 | 180 | 158 | 22 | 12% |
| 02:40 | 175 | 170 | 5 | 3% |
| 03:00 | 151 | 155 | −4 | −3% |
| 03:20 | 156 | 142 | 14 | 9% |

The solid lines in FIG. 8 mark an acceptance region defined as 20% above and below the reference glucose level. As will be appreciated by one of ordinary skill in the art, the band between the solid lines corresponds to the “A zone” of the standard Clarke Error Grid (see Clarke et al., supra). As shown in Table 4 and FIG. 8, all the estimates glucose levels fall within the acceptance region of ±20%.

### Subject No. 2

Table 5 below summarizes the reference glucose history of subject No. 2, the entries of each parameter and the calculated correlation score of each parameter.

TABLE 5

| | Time | | | | | | | | | | |
|---|---|---|---|---|---|---|---|---|---|---|---|
| Parameter | 0 | 20 | 01:00 | 01:20 | 01:40 | 02:00 | 02:20 | 02:40 | 03:00 | 03:20 | score |
| Base | 121 | 121 | 124 | 126 | 123 | 123 | 125 | 125 | 125 | 124 | 0.68 |
| As | 18 | 20.5 | 31 | 26 | 32 | 30 | 27 | 29 | 31 | 25 | 0.61 |
| heart rate | 66 | 64 | 64 | 65 | 65 | 63 | 60 | 59 | 60 | 65 | −0.61 |
| T | 3 | 1 | 3 | 20.5 | 26 | 0 | 6 | 21 | 16 | 1 | 0.30 |
| beta | 13.3 | 17.9 | 18 | 13.1 | 15.6 | 16.3 | 16.3 | 17.9 | 13.4 | 15.3 | 0.04 |
| XV | 23.9 | 21.1 | 43.9 | 34.9 | 31.8 | 33.3 | 36.2 | 31.9 | 33.7 | 30 | 0.33 |
| Alfa | 4.2 | 6.05 | 8.5 | 7 | 7.3 | 8.2 | 8.4 | 8.4 | 8.3 | 7.55 | 0.77 |
| HP | 13 | 14.3 | 22.7 | 22.4 | 30.85 | 24.5 | 18.2 | 19.3 | 26 | 14.1 | 0.33 |
| NG | 30 | 28.5 | 56 | 56 | 57 | 62 | 66 | 64 | 64 | 61.5 | 0.88 |
| gamma | 5 | 6.05 | 9.7 | 7.3 | 11.3 | 9.4 | 8.2 | 8.4 | 9.25 | 7.25 | 0.49 |
| Ad | 8.2 | 9.2 | 14.9 | 15.1 | 21.6 | 17.6 | 13.7 | 15.2 | 19.75 | 9.95 | 0.48 |
| EW | 1.1 | 9.55 | 68 | 103.2 | 101 | 119 | 5.6 | 15.2 | 127.75 | 4.65 | 0.23 |
| Ad-Ai | 0 | 0 | 1.4 | 2.1 | 1.6 | 1.5 | 0 | 1.3 | 1 | 0.7 | 0.29 |
| As/Ad | 231.7 | 224.45 | 208.1 | 166.7 | 148.1 | 170.5 | 197.1 | 197.3 | 159.4 | 255.1 | −0.28 |
| As/XX | 24 | 26.85 | 32.9 | 26.9 | 35.2 | 31.8 | 26.8 | 27.9 | 30.45 | 26.1 | 0.20 |
| As/Av | 172.85 | 143.8 | 154.45 | 119.45 | 127.25 | 147.8 | 154.1 | 156.1 | 129.55 | 149.1 | −0.35 |
| As/Ai | 268.05 | 236 | 261.8 | 194.65 | 191.6 | 227.3 | 263 | 253.8 | 205.95 | 273.7 | −0.14 |
| XH | 36.9 | 24.7 | 22.4 | 22.4 | 22.4 | 22.4 | 22.4 | 25.6 | 23.2 | 19.2 | −0.70 |
| HX | 38.2 | 46.2 | 70.3 | 67.3 | 69.5 | 73.6 | 78.5 | 76.8 | 76.1 | 71.9 | 0.88 |
| reference glucose | 72 | 96 | 101 | 122 | 130 | 140 | 146 | 152 | 153 | 158 | |
| history | | | | | | | | | | | |

The criterion for the calculation of F was the same as for subject No. 1. Three parameters with highest scores were identified for subject No. 2: Base with a correlation score of 0.68, As with a correlation score of 0.61 and HX with a correlation score of 0.88. The following correlation function was obtained for subject No. 2:

F(Base,As,HX)=590.94−4.81378Base−3.52674As+3.389714HX

Table 6 below displays the deviating of F from the reference glucose history.

TABLE 6

| | ref. glucose | | | | estimated | | |
|---|---|---|---|---|---|---|---|
| Time | history | Base | As | HX | glucose | Δ | Δ [%] |
| 0 | 72 | 121 | 18 | 38.2 | 74 | 2 | 2.8% |
| 20 | 96 | 121 | 20.5 | 46.2 | 93 | −3 | −3.1% |
| 01:00 | 122 | 126 | 26 | 70.3 | 121 | −1 | −0.8% |
| 01:20 | 101 | 124 | 31 | 67.3 | 123 | 22 | 21.8% |
| 01:40 | 158 | 124 | 25 | 69.5 | 150 | −8 | −5.1% |
| 02:00 | 152 | 125 | 29 | 73.6 | 147 | −5 | −3.3% |
| 02:20 | 146 | 125 | 27 | 78.5 | 160 | 14 | 9.6% |
| 02:40 | 153 | 125 | 31 | 76.8 | 138 | −15 | −9.8% |
| 03:00 | 140 | 123 | 30 | 76.1 | 142 | 2 | 1.4% |
| 03:20 | 130 | 123 | 32 | 71.9 | 122 | −8 | −6.2% |

The corresponding standard deviation and correlation factor are 13.54 and 0.85, respectively. As shown, one estimate exceeded the predetermined limit of 20%, in agreement with the predetermined criterion for the calculation of F.

Table 7 below presents the values of the parameters Base, As and HX as extracted from the time-dependences obtained from 10 additional cycles of measurements performed for subject No. 2. The right column of Table 7 presents the glucose level as estimated according to the teachings of the present embodiments based on the reference glucose history of subject No. 2 (see Table 5) using the correlation function which is specific to subject No. 2.

TABLE 7

| Time | Base | As | HX | estimated glucose |
|---|---|---|---|---|
| 0 | 121 | 25.5 | 38.2 | 70 |
| 20 | 121 | 24 | 46.2 | 87 |
| 01:00 | 124 | 34.5 | 70.3 | 100 |
| 01:20 | 124 | 27 | 67.3 | 132 |
| 01:40 | 124 | 25 | 69.5 | 153 |
| 02:00 | 125 | 27 | 73.6 | 143 |
| 02:20 | 125 | 29.5 | 78.5 | 132 |
| 02:40 | 124 | 29 | 76.8 | 153 |
| 03:00 | 122 | 28 | 76.1 | 154 |
| 03:20 | 123 | 28 | 71.9 | 127 |

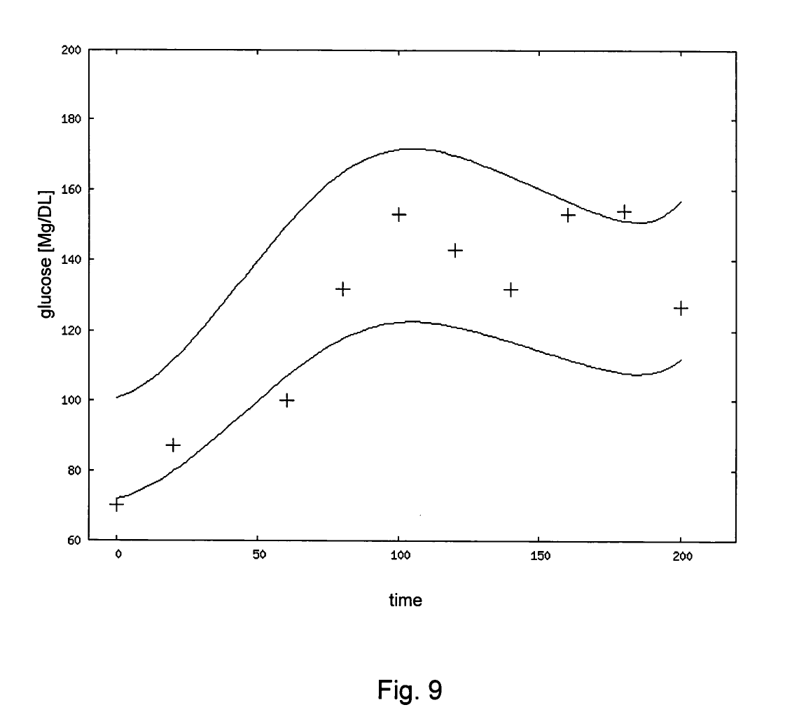

Table 8 below and FIG. 9 compare between the glucose levels of Table 7 as estimated according to the teachings of the present embodiments, and glucose levels measured invasively. The reference glucose levels in Table 8 were not used in the determination of the correlation function.

TABLE 8

| | reference | estimated | | |
|---|---|---|---|---|
| Time | glucose | glucose | Δ | Δ [%] |
| 0 | 90 | 70 | 20 | 22% |
| 20 | 93 | 87 | 6 | 6% |
| 01:00 | 123 | 100 | 23 | 19% |
| 01:20 | 178 | 132 | 46 | 26% |
| 01:40 | 165 | 153 | 12 | 7% |
| 02:00 | 147 | 143 | 4 | 3% |
| 02:20 | 146 | 132 | 14 | 10% |
| 02:40 | 146 | 153 | −7 | −5% |
| 03:00 | 123 | 154 | −31 | −25% |
| 03:20 | 140 | 127 | 13 | 9% |

The solid lines in FIG. 9 mark an acceptance region defined as 20% above and below the reference glucose level. As shown in Table 8 and FIG. 9, the estimated glucose levels at times 0, 01:20 and 03:00 fall outside the acceptance region. The criterion for the calculation of a three variable function was, therefore, not satisfied for subject No. 2. According to a preferred embodiment of the present invention the procedure for this type of subjects is repeated but with shorter intervals of times between successive measurements and/or for more than three variables.

### Subject No. 3

Table 9 below summarizes the reference glucose history of subject No. 3, the entries of each parameter and the calculated correlation score of each parameter.

TABLE 9

| | Time | | | | | | | | | | |
|---|---|---|---|---|---|---|---|---|---|---|---|
| Parameter | 0 | 20 | 01:00 | 01:20 | 01:40 | 02:00 | 02:20 | 02:40 | 03:00 | 03:20 | score |
| Base | 139 | 139 | 138 | 140 | 141 | 142 | 143 | 144 | 143 | 147 | 0.79 |
| As | 14 | 16 | 13 | 14.5 | 14.5 | 18 | 19 | 21 | 16 | 18.5 | 0.53 |
| heart rate | 72 | 73 | 76 | 76 | 78 | 74 | 78 | 77 | 76 | 74 | 0.57 |
| T | 1 | 4 | 4 | 2 | 2 | 0.5 | 0 | 3 | 3 | 2.5 | −0.27 |
| beta | 12.1 | 13.5 | 10.2 | 6.4 | 12.7 | 9.6 | 8 | 7.4 | 12.1 | 7.7 | −0.54 |
| XV | 52.2 | 32.7 | 52.4 | 50.1 | 45.4 | 34.7 | 50 | 36 | 35.1 | 253 | 0.31 |
| Alfa | 3.5 | 3.6 | 2.7 | 2.3 | 3 | 2.2 | 2.4 | 2.3 | 3 | 2.2 | −0.76 |
| HP | 12.3 | 11.8 | 14 | 24.3 | 17.65 | 26 | 35.4 | 41.2 | 20.5 | 32.35 | 0.72 |
| NG | 42.5 | 48 | 37.5 | 27.5 | 37 | 29 | 24 | 22.5 | 38 | 23 | −0.77 |
| gamma | 4.6 | 5.05 | 5.5 | 4.1 | 8.1 | 4.55 | 6 | 4.7 | 6 | 4.25 | 0.11 |
| Ad | 6.8 | 7.85 | 7.45 | 8.65 | 10.3 | 10.6 | 11.7 | 12.7 | 10 | 10 | 0.76 |
| EW | 12.7 | 35.3 | 146.95 | 77.65 | 137.9 | 0 | 74.7 | 157.7 | 182.4 | 162.15 | 0.57 |
| Ad-Ai | 0.7 | 7.1 | 1.3 | 2.3 | 1.2 | 2.1 | 0 | 4.2 | 0 | 0 | −0.56 |
| As/Ad | 213.2 | 206.25 | 174.5 | 167.75 | 135.9 | 166.35 | 162.4 | 161.7 | 170 | 186.7 | −0.66 |
| As/XX | 17.3 | 19.9 | 16.5 | 25.75 | 20.8 | 35.15 | 36.3 | 49.85 | 23.6 | 25.25 | 0.53 |
| As/Av | 216.6 | 158.4 | 176.85 | 138.9 | 168.05 | 140.3 | 166.7 | 120.95 | 144.75 | 236.8 | −0.19 |
| As/Ai | 325.85 | 307.7 | 243 | 283 | 234.8 | 322.5 | 221.75 | 238.35 | 262.05 | 195.7 | −0.59 |
| XH | 24.8 | 16.8 | 25.7 | 18.2 | 24 | 14.8 | 17.6 | 12.1 | 20.3 | 45.2 | 0.14 |
| HX | 56.7 | 57.5 | 51.3 | 37.5 | 49.7 | 34.5 | 32.55 | 27.4 | 46.75 | 26.9 | −0.77 |
| reference glucose | 127 | 111 | 153 | 174 | 177 | 188 | 190 | 191 | 207 | 202 | |
| history | | | | | | | | | | | |

The criterion for the calculation of F was the same as for subject No. 1. Four parameters with highest scores were identified for subject No. 3: Base with a correlation score of 0.79, α with a correlation score of 0.76, Ad with a correlation score of 0.76 and HX with a correlation score of −0.77. The following correlation function was obtained for subject No. 3:

F(Base,α,Ad,HX)=11.39656Base−88.834α+8.19214Ad+4.788743HX−1480.32

Table 10 below displays the deviating of F from the reference glucose history of subject No. 3.

TABLE 10

| | ref. | | | | | | | |
|---|---|---|---|---|---|---|---|---|
| | glucose | | | | | estimated | | |
| Time | history | Base | α | Ad | HX | glucose | Δ | Δ [%] |
| 0 | 127 | 139 | 3.5 | 6.8 | 56.7 | 120 | −7 | −5.5% |
| 20 | 111 | 139 | 3.6 | 7.85 | 57.5 | 124 | 13 | 11.7% |
| 01:00 | 153 | 138 | 2.7 | 7.45 | 51.3 | 159 | 6 | 3.9% |
| 01:20 | 174 | 140 | 2.3 | 8.65 | 37.5 | 161 | −13 | −7.5% |
| 01:40 | 177 | 141 | 3 | 10.3 | 49.7 | 182 | 5 | 2.8% |
| 02:00 | 188 | 142 | 2.2 | 10.6 | 34.5 | 195 | 7 | 3.7% |
| 02:20 | 190 | 143 | 2.4 | 11.7 | 32.55 | 188 | −2 | −1.1% |
| 02:40 | 191 | 144 | 2.3 | 12.7 | 27.4 | 192 | 1 | 0.5% |
| 03:00 | 207 | 143 | 3 | 10 | 46.75 | 189 | −18 | −8.7% |
| 03:20 | 202 | 147 | 2.2 | 10 | 26.9 | 210 | 8 | 4.0% |

The corresponding standard deviation and correlation factor are 13.34 and 0.90, respectively. As shown, no estimated glucose level exceeded the predetermined limit of 20%.

Table 11 below presents the values of the parameters Base, α, Ad and HX as extracted from the time-dependences obtained from 10 additional cycles of measurements performed for subject No. 3. The right column of Table 11 presents the glucose level as estimated according to the teachings of the present embodiments based on the reference glucose history of subject No. 3 (see Table 9) using the correlation function which is specific to subject No. 3.

TABLE 11

| | | | | | estimated |
|---|---|---|---|---|---|
| Time | Base | α | Ad | HX | glucose |
| 0 | 139 | 3.6 | 6.8 | 56.8 | 112 |
| 20 | 138 | 2.2 | 4.8 | 35.2 | 105 |
| 01:00 | 139 | 3.2 | 8.3 | 56.1 | 156 |
| 01:20 | 140 | 2.7 | 9.5 | 45.5 | 171 |
| 01:40 | 141 | 2.8 | 9.3 | 46.25 | 176 |
| 02:00 | 144 | 3.55 | 9.8 | 53 | 180 |
| 02:20 | 143 | 3.35 | 10.9 | 49.8 | 180 |
| 02:40 | 143 | 3.2 | 9.45 | 52.9 | 196 |
| 03:00 | 144 | 2.85 | 9.8 | 43.7 | 197 |
| 03:20 | 149 | 1.55 | 4.4 | 14.4 | 185 |

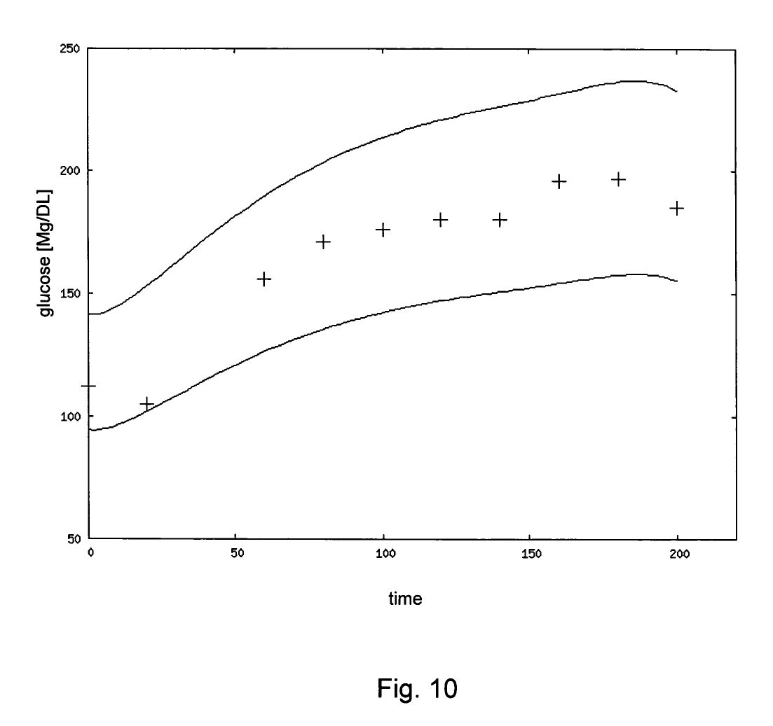

Table 12 below and FIG. 10 compare between the glucose levels of Table 11 as estimated according to the teachings of the present embodiments, and glucose levels measured invasively. The reference glucose levels in Table 12 were not used in the determination of the correlation function.

TABLE 12

| | reference | estimated | | |
|---|---|---|---|---|
| Time | glucose | glucose | Δ | Δ [%] |
| 0 | 118 | 112 | 6 | 5% |
| 20 | 113 | 105 | 8 | 8% |
| 01:00 | 180 | 156 | 24 | 15% |
| 01:20 | 164 | 171 | −7 | −4% |
| 01:40 | 182 | 176 | 6 | 3% |
| 02:00 | 191 | 180 | 11 | 6% |
| 02:20 | 184 | 180 | 4 | 2% |
| 02:40 | 189 | 196 | −7 | −4% |
| 03:00 | 206 | 197 | 9 | 5% |
| 03:20 | 194 | 185 | 9 | 5% |

The solid lines in FIG. 10 mark an acceptance region defined as 20% above and below the reference glucose level. As shown in Table 12 and FIG. 10, all estimated glucose levels fall within the acceptance region.

### Example 2

### Clinical Trials

A clinical study was performed on 16 adult subjects at Assaf Harofe Medical Center, Israel.

### Methods

For each subject, a reference glucose history was recorded at least once and a corresponding subject-specific correlation function was determined according to the teachings of preferred embodiments of the present invention. The predetermined criterion for the calculation of the subject-specific correlation function was that no more than two values of the correlation function will deviate from the reference glucose history of the subject under study by more than 20%. One subject, for which the criterion was not satisfied, was rejected.

Data were acquired from the remaining 15 subjects: 4 diabetics of ages 60-65 (3 males, 1 female), 5 healthy adults of ages 26-32 (3 males, 2 females) and 6 healthy adults of ages 55-65 (3 males, 3 females).

For each subject, reference blood glucose levels were obtained invasively using FreeStyle™ blood glucose monitoring system, and estimated glucose levels were calculated based on the reference glucose history of the subject under study and using the subject-specific correlation function. About 10 reference and about 20 estimated glucose levels were recorded for each subject. The obtained glucose levels were displayed on a scatter plot of estimated glucose level versus reference glucose levels. The entire dataset included 279 points.

The scatter plot was superimposed on a Clarke Error Grid, which is a grid divided into five zones, denoted A, B, C, D, and E, that assess the measurement accuracy on the basis of validity of the corresponding clinical decision (see Clarke et al., supra).

The “A zone” of the Clarke Error Grid is typically defined as the zone for which the estimated levels deviate by no more than 20% from the reference levels, and the “B zone” is typically defined as the zone for which the estimated levels deviate by more than 20% from the reference levels but treatment decisions made based on the estimated levels of glucose would not jeopardize or adversely affect the subject. Generally, data points that are in the “A” and “B” zones of the Clarke Error Grid are deemed acceptable, because they present estimate glucose levels close to the reference blood glucose level or estimated levels that are less accurate but would not lead to wrong clinical intervention. The performance of the tested technique is considered to be better when the percentage of data points in the “A zone” increases and the percentage of data points in the “B zone” decreases. The “C”, “D” and “E” zones of the Clarke Error Grid are typically defined as the zones in which the estimated levels significantly deviate from the reference values, and treatment decisions based on these estimates may well be harmful to a patient.

According to the FDA stipulation, for a technique or system to be FDA approved, 80% of the data points should fall within the “A zone” of the Clarke Error Grid, 20% of the data points should fall within the “B zone”, and no data point is allowed to fall within the “C”, “D” or “E” zone.

### Results

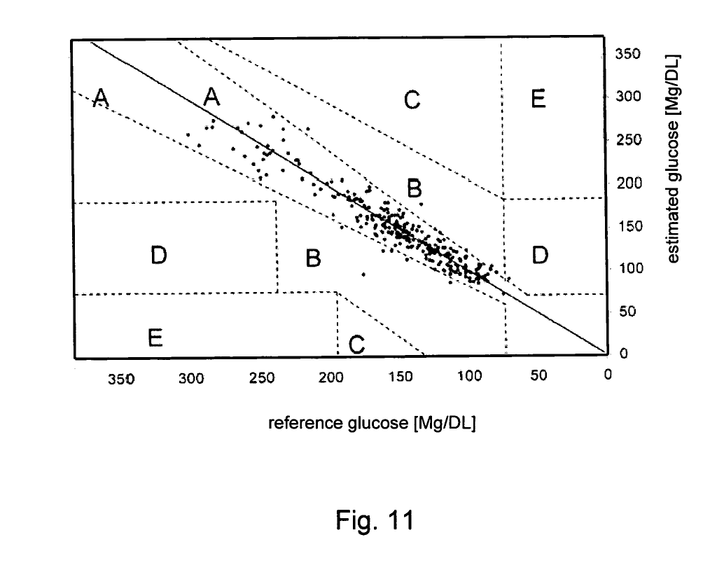

FIG. 11 is a scatter plot showing estimated glucose level versus reference glucose levels, superimposed on a Clarke Error Grid. The mean absolute deviation was 7.9 Mg/DL (5.3%). 268 data points (96.1%) fall in the “A zone” and 11 data points (3.9%) fall in the “B zone” of the Clarke Error Grid. No data point (0.0%) falls within the “C”, “D” or “E” zone, in accordance with the FDA stipulation. This example thus demonstrates that the technique of the present embodiments provides an accurate and reliable non-invasive glucose level monitoring.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of determining a subject-specific correlation function correlating an electrical quantity characterizing a section of a subject body to a glucose level of the subject, the method comprising:
non-invasively measuring the electrical quantity, so as to provide a time-dependence of said electrical quantity over a predetermined time-period;
measuring the glucose level of the subject a plurality of times, thereby providing a series of glucose levels;
using said time-dependence for extracting a plurality of parameters characterizing said time-dependence, wherein said plurality of parameters comprises at least four parameters; and
performing a statistical analysis so as to correlate said series of glucose levels to at least one of said plurality of parameters;
thereby determining the subject-specific correlation function.

2. The method of claim 1, wherein said subject-specific correlation function is defined over a plurality of variables, each variable of said plurality of variables corresponding to a different parameter of said plurality of parameters.

3. The method of claim 2, wherein said plurality of variables are respectively weighted by a plurality of subject-specific coefficients.

4. The method of claim 2, wherein at least one variable of said plurality of variables is powered by a subject-specific power.

5. A method of estimating the glucose level of a subject having a glucose level history, the method comprising calculating a subject-specific correlation function describing the glucose level history, and using said subject-specific correlation function for estimating the glucose level of the subject;
said subject-specific correlation function being defined over a plurality of variables, each corresponding to a different parameter characterizing a time-dependence of an electrical quantity over a predetermined time period being correlated to a heart rate of the subject.

6. A method of monitoring the glucose level of a subject having a glucose level history, comprising:
non-invasively measuring an electrical quantity from a section of the subject body so as to provide a time-dependence of said electrical quantity over a predetermined time-period being correlated to a heart rate of the subject;
using said time-dependence for extracting a plurality of parameters characterizing said time-dependence;
calculating a subject-specific correlation function describing the glucose level history, said subject-specific correlation function being defined over a plurality of variables, each corresponding to a different parameter of said plurality of parameters; and
using said subject-specific correlation function for estimating the glucose level of the subject;
thereby monitoring the glucose level of the subject.

7. The method of claim 6, wherein said plurality of variables are respectively weighted by a plurality of subject-specific coefficients.

8. The method of claim 7, wherein at least one variable of said plurality of variables is powered by a subject-specific power.

9. The method of claim 6, further comprising testing the accuracy of said subject-specific correlation function according to a predetermined accuracy criterion, and, if said predetermined accuracy criterion is not satisfied then updating said subject-specific correlation function.

10. The method of claim 6, further comprising updating said subject-specific correlation function at least once.

11-14. (canceled)

15. A system for determining a subject-specific correlation function correlating an electrical quantity characterizing a section of a subject body to a glucose level of the subject, the system comprising:
(a) a glucose level input unit configured for receiving a series of glucose levels;
(b) a non-invasive measuring device operable to measure and record the electrical quantity, so as to provide a time-dependence of said electrical quantity over a predetermined time-period; and
(c) a processing unit communicating with said non-invasive measuring device, and comprising:
(i) an extractor, communicating with said non-invasive measuring device and being operable to extract a plurality of parameters characterizing said time-dependence, wherein said plurality of parameters comprises at least four parameters; and
(ii) a correlating unit, communicating with said extractor and being supplemented with statistical analysis software configured to correlate said series of glucose levels to at least one of said plurality of parameters, thereby to determine the subject-specific correlation function.

16. Apparatus for estimating the glucose level of a subject having a glucose level history, the apparatus comprising:
a correlation function calculator, operable to calculate a subject-specific correlation function describing the glucose level history, and to estimate the glucose level of the subject based on said subject-specific correlation function, wherein said subject-specific correlation function is defined over a plurality of variables, each corresponding to a different parameter characterizing a time-dependence of an electrical quantity over a predetermined time period being correlated to a heart rate of the subject; and
an output unit, communicating with said correlation function calculator and configured to output the glucose level of the subject.

17. A monitoring system for monitoring the glucose level of a subject having a glucose level history, the system comprising:
(a) a non-invasive measuring device operable to measure and record an electrical quantity from a section of the subject body, so as to provide a time-dependence of said electrical quantity over a predetermined time-period being correlated to a heart rate of the subject; and
(b) a processing unit, communicating with said non-invasive measuring device and comprising:
(i) an extractor operable to extract a plurality of parameters characterizing said time-dependence,
(ii) a correlation function calculator operable to calculate a subject-specific correlation function describing the glucose level history and to estimate the glucose level of the subject based on said subject-specific correlation function, wherein said subject-specific correlation function is defined over a plurality of variables, each corresponding to a different parameter of said plurality of parameters, and
(iii) an output unit, communicating with said correlation function calculator and configured to output the glucose level of the subject.

18-20. (canceled)

21. The system of claim 17, further comprising an updating unit designed and configured for updating said subject-specific correlation function at least once.

22-34. (canceled)

35. The method of claim 1, wherein said predetermined time-period is correlated to a heart rate of the subject.

36. The method of claim 35, wherein said predetermined time-period equals at least a heart beat cycle of the subject.

37. The method of claim 35, wherein said predetermined time-period equals an integer number of heart beat cycles of the subject.

38-41. (canceled)

42. The method of claim 1, wherein at least one of said plurality of parameters comprises a value of said electrical quantity at a transition point on said time-dependence.

43. The method of claim 1, wherein at least one of said plurality of parameters comprises a ratio between two values of said electrical quantity, said two values corresponding to different transition points on said time-dependence.

44. The method of claim 1, wherein at least one of said plurality of parameters comprises a difference between two values of said electrical quantity, said two values corresponding to different transition points on said time-dependence.

45. (canceled)

46. The method of claim 1, wherein at least one of said plurality of parameters comprises a time-interval corresponding to a transition point on said time-dependence.

47. The method of claim 1, wherein at least one of said plurality of parameters comprises a time-derivative of said time-dependence.

48. The method of claim 1, wherein at least one of said plurality of parameters comprises an average time-derivative of at least a segment of said time-dependence.

49. The method of claim 1, wherein at least one of said plurality of parameters comprises a slope along a segment of said time-dependence.

50-52. (canceled)
